(19) United States
(12) Patent Application Publication
(10) Pub. No.: US 2020/0155851 A1
(43) Pub. Date: May 21, 2020

(54) KELVIN-CONNECTION SCHEME FOR DIAGNOSTIC CAPABILITY IN A NEUROSTIMULATOR

(71) Applicant: ADVANCED NEUROMODULATION SYSTEMS, INC., Plano, TX (US)

(72) Inventors: Steven Boor, Plano, TX (US); Daran DeShazo, Lewisville, TX (US)

(21) Appl. No.: 16/195,502

(22) Filed: Nov. 19, 2018

(51) Int. Cl. A61N 1/36; A61N 1/05

(52) U.S. Cl. CPC A61N 1/36125; A61N 1/36142; A61N 1/36062; A61N 1/0551

(57) ABSTRACT

A Kelvin connection scheme, apparatus and method for effectuating residual voltage measurements with respect to one or more electrodes of a lead system associated with an implantable medical device (IMD). In one example implementation, terminals of at least one DC blocking stimulation capacitor or at least one AC-coupling sense capacitor associated with at least one inactive electrode of the lead system are used as one Kelvin connection terminal of a measurement circuit path whereas a counter Kelvin connection terminal with respect to a selection of at least one active electrode is effectuated across the electrode/tissue interface using either at least one DC blocking stimulation capacitor or at least one AC-coupling sense capacitor provided therewith.

### TECHNICAL FIELD

The present disclosure generally relates to implantable pulse generators and circuitry used in association therewith. More particularly, and not by way of any limitation, the present disclosure is directed to a Kelvin-connection scheme for facilitating diagnostic measurement capability in neurostimulation systems (NS) including but not limited to spinal cord stimulation (SCS) systems.

### BACKGROUND

The use of electronic stimulation systems to control pain or other indications, or to otherwise provide therapy, by nerve or muscle stimulation has been in use for a number of years. For example, spinal cord stimulation (SCS) is a technique that has been used for pain management since the 1960s. Stimulation systems may also be used in stimulating areas other than the spinal cord, such as for deep brain stimulation, muscle stimulation, etc.

Stimulation systems often comprise a pulse generator coupled to one or more therapy delivery leads having a plurality of electrodes disposed in an area in which neurostimulation is desired. Alternatively, stimulation systems may comprise a micro-stimulation system in which a small implantable housing having electrodes thereon includes a pulse generator, wherein the entire micro-stimulation system is disposed in an area in which neurostimulation is desired. Of course, all or a portion of a stimulation system need not be implanted into a body to provide a desired therapy.

A stimulation system pulse generator may be provided in various configurations, such as a totally implanted pulse generator (IPG) or a radio frequency (RF)-based system. A typical IPG configuration comprises a surgically implanted, internally-powered pulse generator and a multi-electrode lead. A typical RF system configuration comprises a surgically implanted, passive receiver and a transmitter which is worn externally. In operation, the transmitter communicates, through an RF signal, to the implanted receiver to provide stimulation energy and control.

In an SCS application, electrodes which are used with an example pulse generator, such as any of the foregoing pulse generators, to deliver a particularized electric field via stimulation to a specific region of the spinal cord or surrounding tissue are considered as the “active” electrodes of the IPG for therapy delivery; unused or “inactive” electrodes are the ones not used for stimulation therapy. Applying such an electric field with the active electrodes across one or more nerve bundles and/or nerve roots, if properly directed and produced at the necessary levels, can “mask” certain forms of chronic pain in a phenomenon referred to as “paresthesia”. Similarly, applying an electric field across other tissue, such as muscle or brain matter, near which such electrodes are disposed may provide a desired therapy. The focus, characteristics and intensity of the generated electric field are determined by the electrode configuration (the polarity, if any, assumed by each electrode) and the properties of an electric pulse waveform, which may generally include a stimulation frequency, a stimulation pulse width, a stimulation amplitude, discharge method, and phase information, etc. (collectively “stimulation settings” or “stimsets”).

Implantation of all or a portion of a stimulation system, e.g., a stimulation system including a fully implanted IPG or a RF system receiver/transmitter, necessarily requires a neurostimulation patient to undergo an implantation surgery. Additionally, routing a lead subdermally between an implanted pulse generator and the tissue area to be stimulated typically requires a relatively invasive procedure, such as a tunneling procedure. Likewise, explanting all or a portion of a stimulation system requires a neurostimulation patient to again undergo the trauma of surgery.

Chronically implantable electrical stimulation mechanisms have been the focus of advanced physiological engineering research for the past few decades. With the advent of microelectronics, it has become imperative to look into the criticality of safe functional electrical stimulation for large electrode arrays since stimulation electrode characteristics can change due to electrode dissolution/deterioration during prolonged use. Structural damage can occur if there is exposure to electrode potential much higher than applicable electrochemical windows associated with a tissue interface. Moreover, with large stimulation arrays employed in certain applications, monitoring the status of different electrodes becomes challenging.

Whereas advances in IPG systems for use with NS systems continue to grow apace, several lacunae remain, thereby requiring further innovation as will be set forth hereinbelow.

### SUMMARY

It will therefore be appreciated that minimizing a residual voltage left on electrodes after the stimulation of tissue in a patient's body plays a critical role in maintaining patient safety and electrode integrity. Accordingly, the steady-state residual voltages on the individual electrode/patient interfaces after stimulation must be kept well under certain threshold levels, which can otherwise introduce chemical reactions that can either physically damage the electrodes, cause patient discomfort, and/or cause detrimental biological reactions in patients. Hereinafter in this patent application, the term “residual voltage” is to be interpreted, at least in some embodiments, to mean after a complete stimulation/discharge cycle of therapy, or as a steady-state condition as a consequence of the stimulation therapy delivery.

Embodiments of the present patent disclosure are broadly directed to implantable pulse generators or other medical devices (IPG/IMD), systems and associated circuitry wherein various types of Kelvin connection schemes and apparatuses are provided for effectuating residual voltage measurements with respect to one or more electrodes of a lead system associated with an IMG/IMD system. In an example implementation, terminals of a DC blocking stimulation capacitor or an AC-coupling sense capacitor associated with an inactive electrode of the lead system may be configured as one Kelvin connection terminal of a measurement circuit path whereas a counter Kelvin connection terminal with respect to a select active electrode is effectuated across the electrode/tissue interface using either a DC blocking stimulation capacitor or an AC-coupling sense capacitor provided therewith.

In one aspect, an embodiment of the present disclosure is directed to an implantable pulse generator or other medical device (IPG/IMD) for stimulating biological tissue, comprising, inter alia, a power supply module, a processing unit and an implantable lead system comprising a plurality of electrodes adapted to stimulate a biological tissue responsive to instructions generated by the processing unit in association with a pulse switching module, wherein the plurality of electrodes include at least one inactive electrode and at least one active electrode. Diagnostic circuitry coupled to the processing unit and the pulse switching module is advantageously configured to: utilize/configure one of at least one direct current (DC) blocking stimulation capacitor (C_{DC}) terminal and at least one alternating current (AC) coupling sense capacitor (C_{SENSE}) terminal of the at least one inactive electrode of the implantable lead system as a first Kelvin connection terminal for a residual voltage measurement with respect to a selection of the at least one active electrode of the implantable lead system; utilize/configure a terminal of at least one alternating current (AC) coupling sense capacitor (C_{SENSE}) coupled to the selection of the at least one active electrode as a second Kelvin connection terminal for the residual voltage (RV) measurement; and electrically couple a voltage measurement circuit (external and/or internal) to the first and second Kelvin connection terminals to measure a residual voltage associated with the selection of the at least one active electrode that is accumulated across at least one double-layer (DL) capacitance (C_{DL}) associated with an electrode/tissue interface of the selection of the at least one active electrode.

In another aspect, an embodiment of method operative with an IPG/IMD system configured to supply stimulation to a biological tissue is disclosed. The claimed embodiment comprises, inter alia, configuring at least one of a plurality of electrodes of an implantable lead system of the pulse generator as an active electrode operative to apply stimulation therapy to the biological tissue responsive to instructions generated by a processing unit in association with a pulse switching module; configuring at least one electrode of the implantable lead system as an inactive electrode; utilizing a C_{DC} terminal of at least one inactive electrode of the implantable lead system as a first terminal for a residual voltage measurement with respect to a selection of the at least one active electrode of the implantable lead system; utilizing at least one C_{DC} terminal of the selection of the at least one active electrode of the implantable lead system as a second terminal for the RV measurement; and electrically coupling a voltage measurement circuit (external and/or internal) to the first and second terminals to measure a residual voltage associated with the selection of the at least one active electrode, wherein the first terminal is operative to provide a Kelvin connection path with respect to the at least one inactive electrode for the voltage measurement circuit such that the residual voltage is obtained as a sum of a first residual voltage (RV) component accumulated across the at least one C_{DC} capacitor coupled to the selection of the at least one active electrode and a second residual voltage (RV) component accumulated across at least one double-layer (DL) capacitance (C_{DL}) associated with an electrode/tissue interface of the selection of the at least one active electrode.

Skilled artisans will recognize that example embodiments of the present patent disclosure may therefore be advantageously configured so as to allow for DC blocking stimulation capacitors connected to the stimulation electrodes to continue to be used for maintaining patient safety while also allowing for other unused stimulation electrodes and biosensing input AC-coupling capacitors to “Kelvin-connect” to both/either sides of the electrode/patient interface (EPI) for the measurement of residual voltages on individual electrodes. It will be seen that an embodiment may also involve any combination of any subset of the active electrodes and any subset of the unused/inactive electrodes in a Kelvin connection path on either side of the EPI interface for an RV measurement.

Example embodiments may also be configured to advantageously provide a stimulation diagnostic capability that allow residual voltage measurements to be made on each electrode/patient interface before and after stimulation, thereby enabling the IPG/IMD system to provide an optimal amount of discharge for each electrode. Accordingly, such optimization can ensure both patient safety and electrode integrity, minimize patient discomfort, as well as potentially help improve the device battery longevity, thereby resulting in improved patient therapy.

Additional/alternative features and variations of the embodiments will be apparent in view of the following description and accompanying Figures.

### BRIEF DESCRIPTION OF THE DRAWINGS

Embodiments of the present disclosure are illustrated by way of example, and not by way of limitation, in the Figures of the accompanying drawings in which like references indicate similar elements. It should be noted that different references to “an” or “one” embodiment in this disclosure are not necessarily to the same embodiment, and such references may mean at least one. Further, when a particular feature, structure, or characteristic is described in connection with an embodiment, it is submitted that it is within the knowledge of one skilled in the art to effectuate such feature, structure, or characteristic in connection with other embodiments whether or not explicitly described.

The accompanying drawings are incorporated into and form a part of the specification to illustrate one or more exemplary embodiments of the present disclosure. Various advantages and features of the disclosure will be understood from the following Detailed Description taken in connection with the appended claims and with reference to the attached drawing Figures in which:

FIG. 1A depicts an example biostimulation system wherein one or more embodiments of a diagnostic scheme of the present disclosure may be practiced for effectuating residual voltage (RV) measurements using a Kelvin connection arrangement in accordance with the teachings herein;

FIG. 1B depicts a pulse generator portion having diagnostic circuitry and associated lead electrode capacitor arrangement according to an embodiment of the present disclosure;

FIG. 2A depicts an example circuit diagram for facilitating RV measurements using one type of Kelvin connection paths according to an embodiment of the present disclosure;

FIG. 2B depicts an example circuit diagram for facilitating RV measurements using a combination of Kelvin connection paths according to another embodiment of the present disclosure;

FIGS. 3A-3C depict flowcharts illustrative of blocks, steps and/or acts that may be (re)combined in one or more arrangements for effectuating RV measurements according to an embodiment of the present disclosure; and

FIG. 4 illustrates an example spinal cord stimulation (SCS) therapy application involving a pulse generator and associated lead system having a plurality of electrodes wherein diagnostic RV measurements may be obtained using an embodiment of the present disclosure.

### DETAILED DESCRIPTION

In the description herein for embodiments of the present disclosure, numerous specific details are provided, such as examples of circuits, devices, components and/or methods, to provide a thorough understanding of embodiments of the present disclosure. One skilled in the relevant art will recognize, however, that an embodiment of the disclosure can be practiced without one or more of the specific details, or with other apparatuses, systems, assemblies, methods, components, materials, parts, and/or the like. In other instances, well-known structures, materials, or operations are not specifically shown or described in detail to avoid obscuring aspects of embodiments of the present disclosure. Accordingly, it will be appreciated by one skilled in the art that the embodiments of the present disclosure may be practiced without such specific components. It should be further recognized that those of ordinary skill in the art, with the aid of the Detailed Description set forth herein and taking reference to the accompanying drawings, will be able to make and use one or more embodiments without undue experimentation.

Additionally, terms such as “coupled” and “connected,” along with their derivatives, may be used in the following description, claims, or both. It should be understood that these terms are not necessarily intended as synonyms for each other. “Coupled” may be used to indicate that two or more elements, which may or may not be in direct physical or electrical contact with each other, co-operate or interact with each other. “Connected” may be used to indicate the establishment of communication, i.e., a communicative relationship, between two or more elements that are coupled with each other. Further, in one or more example embodiments set forth herein, generally speaking, an electrical element, component or module may be configured to perform a function if the element may be programmed for performing or otherwise structurally arranged to perform that function.

Some embodiments described herein may be particularly set forth with respect to an implantable pulse generator (IPG) for generating electrical stimulation for application to a desired area of a body or tissue based on a suitable stimulation therapy application, such as a spinal cord stimulation (SCS) system. However, it should be understood that example circuitry and methods of operation disclosed herein are not limited thereto, but have broad applicability, including but not limited to different types of implantable devices such as neuromuscular stimulators and sensors, dorsal root ganglion (DRG) stimulators, deep brain stimulators, cochlear stimulators, retinal implanters, muscle stimulators, tissue stimulators, cardiac stimulators, gastric stimulators, and the like, including other bioelectrical sensors and sensing systems, which may be broadly referred to as “biostimulation” applications and/or implantable medical devices (IMDs) for purposes of the present disclosure. Moreover, example circuitry and methods of operation disclosed herein are not limited to use with respect to an IPG or any particular form of IPG. For example, some embodiments may be implemented with respect to a fully implantable pulse generator, a radio frequency (RF) pulse generator, an external pulse generator, a micro-implantable pulse generator, inter alia.

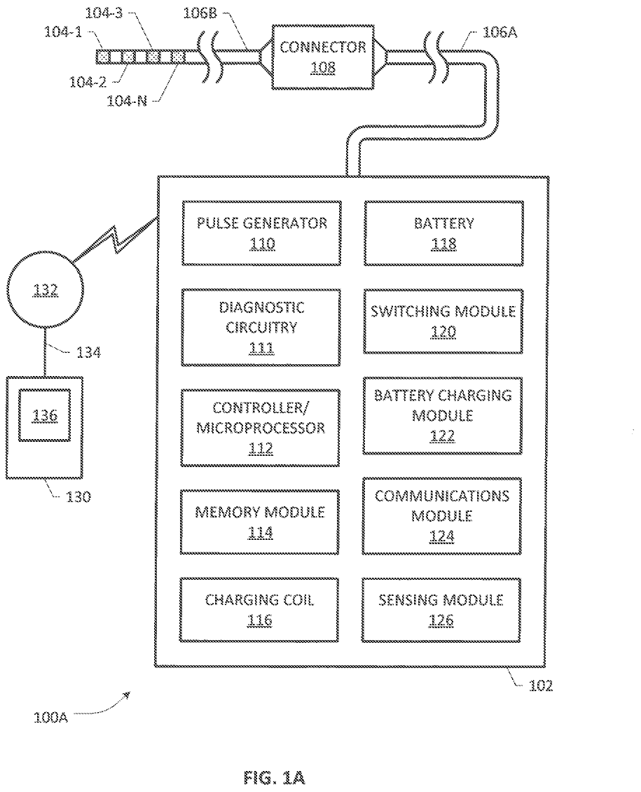

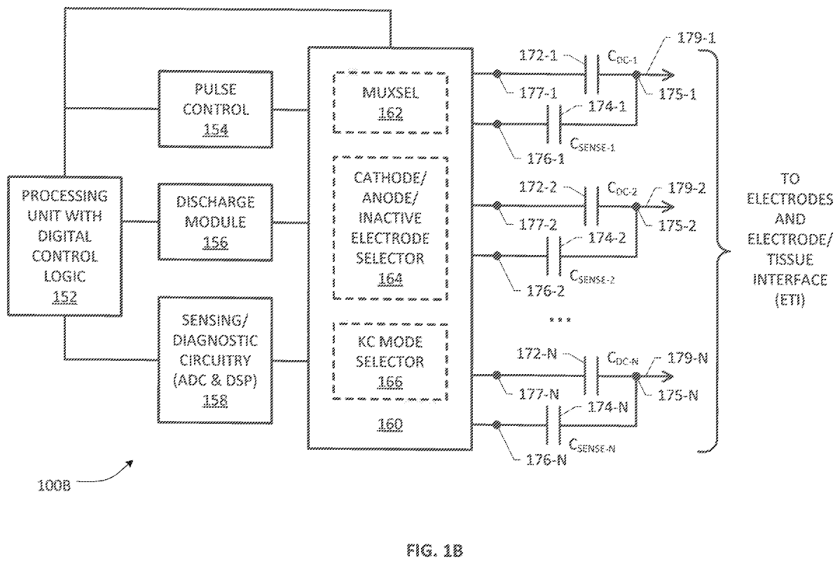

Referring to FIG. 1A in particular, depicted therein is a biostimulation system or IMD 100A wherein one or more embodiments of a diagnostic scheme of the present patent disclosure that may be practiced for effectuating residual voltage measurements using a Kelvin connection arrangement in accordance with the teachings herein. By way of providing a generalized contextual application, an overall description of system 100A is set forth immediately as follows. Broadly, system 100A may be adapted to stimulate spinal cord tissue, peripheral nerve tissue, deep brain tissue, DRG tissue, cortical tissue, cardiac tissue, digestive tissue, pelvic floor tissue, or any other suitable biological tissue of interest within a patient's body, as noted above. System 100A includes an implantable pulse generator (IPG) 102 that comprises a diagnostic circuit module 111 adapted to effectuate Kelvin connections with one or more electrodes of an implantable lead system for measuring residual voltages associated with the electrodes as will be set forth in additional detail hereinbelow. In one example embodiment, IPG 102 may be implemented as having a metallic housing or can that encloses a controller/processing block or module 112, pulse generating circuitry 110, a charging coil 116, a battery 118, a far-field and/or near field communication block or module 124, battery charging circuitry 122, switching circuitry 120, sensing circuitry 126, a memory module 114, and the like. Controller/processor module 112 typically includes a microcontroller or other suitable processor for controlling the various other components of the IPG device 102. Software/firmware code may be stored in memory 114 of IPG 102, which may be integrated with the controller/processor module 112, and/or other suitable application-specific storage components (not particularly shown in this FIG.) for execution by the microcontroller or processor 112 and/or other programmable logic blocks to control the various components of the device for purposes of an embodiment of the present patent disclosure.

In one arrangement, IPG 102 may be coupled to a separate or an attached extension component 106A for providing electrical and physical connectivity to an implantable lead system 106B via a lead connector 108, wherein a distal end of the lead 106B includes a plurality of electrodes 104-1 to 104-N. Where the extension component 106A is provided as a separate component, the extension component 106A may connect with a “header” portion of IPG 102 as is known in the art. If the extension component 106A is integrated with IPG 102, internal electrical connections may be made through respective conductive components. In general, electrical pulses are generated by the pulse generating circuitry 110 under the control of processing block 112, and are provided to the switching circuitry 120 that is operative to selectively connect to electrical outputs of the IPG device, which are ultimately coupled to the electrodes 104-1 to 104-N at a distal end of the lead system 106B via respective electrical conductive traces.

In one arrangement, lead electrodes 104-1 to 104-N may be positioned along an axis of the lead 106B, with an angular offset such that the lead electrodes 104-1 to 104-N do not overlap. The lead electrodes 104-1 to 104-N may be in the shape of a ring such that each lead electrode continuously covers the circumference of the exterior surface of the lead 106B. Typically, the lead electrodes 104-1 to 104-N are separated from each other by non-conducting portions of the lead 106B, which electrically isolate each lead electrode 104-1 to 104-N from an adjacent lead electrode 104-1 to 104-N. The non-conducting portions of the lead 106B may include one or more insulative materials and/or biocompatible materials to allow the lead 106B to be implantable within the patient. Non-limiting examples of such materials include polyimide, polyetheretherketone (PEEK), polyethylene terephthalate (PET) film (also known as polyester or Mylar), polytetrafluoroethylene (PTFE) (e.g., Teflon), or parylene coating, polyether bloc amides, polyurethane, or the like compositions.

Additionally or alternatively, electrodes 104-1 to 104-N may be in the shape of a split or non-continuous ring such that the stimulation pulse(s) may be emitted in a manner so as to create an electric field emanating in an outward radial direction adjacent to the lead electrodes 104-1 to 104-N. Examples of lead electrodes 104-1 to 104-N and associated fabrication processes are disclosed in one or more of the following: (i) U.S. Patent Application Publication No. 2011/0072657, entitled, “METHOD OF FABRICATING STIMULATION LEAD FOR APPLYING ELECTRICAL STIMULATION TO TISSUE OF A PATIENT”; and (ii) U.S. Patent Application Publication No. 2018/0008821, entitled, “IMPLANTABLE THIN FILM DEVICES”, each of which is incorporated herein by reference.

It should be noted the lead electrodes 104-1 to 104-N may be in various other formations, for example, in a planar formation, in an array or grid, etc. on a paddle structure as disclosed in U.S. Patent Application Publication No. 2014/0343564, entitled, “PADDLE LEADS FOR NEUROSTIMULATION AND METHOD OF DELIVERYING THE SAME”, which is incorporated herein by reference.

In one arrangement, the lead system 106B (as well as extension 106A where provided) may comprise a lead body of insulative material encapsulating a plurality of conductors within the material that extend from a proximal end (that is proximate to IPG 102) to the distal end of the lead body containing the lead electrodes 104-1 to 104-N. The conductors or conductive traces are operative to electrically couple the lead electrodes 104-1 to 104-N to a corresponding plurality of terminals (not shown) of the lead system 106A/B. In general, the terminals are adapted to receive electrical pulses from the pulse generation and switching circuitry of IPG 102, which are propagated via the corresponding conductive traces to at least a portion of the lead electrodes 104-1 to 104-N that are adapted to apply the pulses to a desired stimulation target of the patient depending on the particular stimulation therapy application. Also, sensing of physiological or bioelectrical signals may occur through the lead electrodes 104-1 to 104-N, corresponding conductors, and associated terminals. By way of illustration, an example embodiment of the stimulation system 100A may be provided with a plurality of lead electrodes 104-1 to 104-N comprising four electrodes, eight electrodes, etc., although any suitable number of electrodes (as well as corresponding conductive traces and terminals) may be provided in a lead system. Additionally or alternatively, various sensors (e.g., a position detector, temperature sensor, one or more electrochemical sensors, a radiopaque fiducial, etc.) may be located near the distal end of the lead 106B and electrically coupled to terminals through associated conductors within the lead body.

Although not required for all embodiments, the lead body of the implantable lead system 106A/106B may be fabricated to flex and elongate upon implantation or advancing within or relative to the tissue (e.g., nervous tissue) of the patient towards the stimulation target to account for movement of the patient during or after implantation. Fabrication techniques and material characteristics for “body compliant” leads are disclosed in greater detail in U.S. Pat. No. 9,844,661, entitled “COMPLIANT ELECTRICAL STIMULATION LEADS AND METHODS OF FABRICATION”, which is incorporated herein by reference.

An example implementation of the components within IPG 102, such as, e.g., processor and associated charge control circuitry for an IPG, is described in U.S. Pat. No. 7,571,007, entitled “SYSTEMS AND METHODS FOR USE IN PULSE GENERATION”, which is incorporated herein by reference. An example implementation of circuitry for recharging a rechargeable battery (e.g., battery charging circuitry 122) of an IPG using inductive coupling and external charging circuits is described in U.S. Pat. No. 7,212,110, entitled “IMPLANTABLE DEVICE AND SYSTEM FOR WIRELESS COMMUNICATION”, which is incorporated herein by reference. Still further, an example implementation of “constant current” pulse generating circuitry (e.g., at least a portion of pulse generating circuitry 110) is provided in U.S. Patent Application Publication No. 2006/0170486 entitled “PULSE GENERATOR HAVING AN EFFICIENT FRACTIONAL VOLTAGE CONVERTER AND METHOD OF USE”, which is incorporated herein by reference. One or multiple sets of such circuitry may be provided within IPG 102 operating in association with a current control module for providing stimulation across a select number of electrodes. Different stimulation pulses on different lead electrodes selected from electrodes 104-1 to 104-N may be generated using consecutively generated pulses according to a “multi-stimset program” as is known in the art. Complex pulse parameters may be employed such as those described in U.S. Pat. No. 7,228,179, entitled “METHOD AND APPARATUS FOR PROVIDING COMPLEX TISSUE STIMULATION PATTERNS”, and International Patent Publication Number WO 2001/093953, entitled “NEUROMODULATION THERAPY SYSTEM”, which are incorporated herein by reference. Alternatively, multiple sets of such stimulation circuitry may be employed to provide high frequency pulse patterns (e.g., tonic stimulation waveform, burst stimulation waveform, and the like) that include generated and delivered stimulation therapy through one or more leads 104-1 to 104-N as is also known in the art. Various sets of parameters may define the pulse characteristics and pulse timing for the pulses applied to the various lead electrodes as is known in the art. Although constant current pulse generating circuitry is contemplated for some embodiments, any other suitable type of pulse generating circuitry may be employed such as constant voltage pulse generating circuitry.

In an example implementation of IPG 102, sensing circuitry 126 may be provided, preferably adapted to measure a suitable electric parameter or transduced characteristic (e.g., voltage, current, capacitance, etc.) over a configurable time associated with the stimulation target or tissue through at least one of the electrodes proximate to the stimulation target, e.g., electrodes configured to operate as biosensing inputs, wherein such “sensing” electrodes may be coupled to the sensing circuitry 126 via suitable alternating current (AC)-coupling capacitors. In an example embodiment, the sensing circuitry 126 may measure an evoked compound activation potential (ECAP) waveform from an Aβ sensory fiber or spinal cord. Optionally, the sensing circuitry 126 may store the measured/sensed electric data in memory 114. Furthermore, the diagnostic circuitry 111 may be configured to interoperate with the sensing circuitry 126 and pulse generation and switching functionalities of the IPG device 102 for effectuating diagnostic RV measurements as will set forth below in additional detail.

An external device 130 may be implemented to charge/recharge the battery 118 of IPG 102 (although a separate recharging device could alternatively be employed), to access memory 114, and/or to program or reprogram IPG 102 with respect to the stimulation set parameters including pulsing specifications while implanted within the patient. In alternative embodiments, however, separate programmer devices may be employed for charging and/or programming the IPG 102 device and/or any programmable components thereof. An example embodiment of the external device 130 may be a processor-based system that possesses wireline and/or wireless communication capabilities, e.g., a tablet, smartphone, laptop computer, handheld computer, a personal digital assistant (PDA), or any smart wearable device and smart digital assistant device, etc. Software may be stored within a non-transitory memory of the external device 130, which may be executed by the processor to control the various operations of the external device 130. A connector or “wand” 134 may be electrically coupled to the external device 130 through suitable electrical connectors (not specifically shown), which may be electrically connected to a telemetry component 132 (e.g., inductor coil, RF transceiver, etc.) at the distal end of wand 134 through respective communication links that allow bi-directional communication with IPG 102. Optionally, in some embodiments, the wand 134 may comprise one or more temperature sensors for use during charging operations.

In general operation, a user (e.g., a doctor, a medical technician, or the patient) may initiate communication with IPG 102 by placing the wand 134 proximate to the stimulation system 100A. Preferably, the placement of the wand 134 allows the telemetry system to be aligned with the far-field and/or near field communication circuitry 124 of IPG 102. The external device 130 preferably provides one or more user interfaces 136 (e.g., touch screen, keyboard, mouse, buttons, scroll wheels or rollers, or the like), allowing the user to operate IPG 102. The external device 130 may be controlled by the user through the user interface 136, allowing the user to interact with IPG 102, including, e.g., effectuating programmatic control for facilitating diagnostic measurements, dynamically configuring electrodes for different Kelvin connection schemes, etc. as will be set forth further below. Further, the user interface 136 may permit the user to move electrical stimulation along and/or across one or more of the lead(s) 106A using different lead electrode combinations selected from electrodes 104-1 to 104-N, for example, as described in U.S. Patent Application Publication No. 2009/0326608, entitled “METHOD OF ELECTRICALLY STIMULATING TISSUE OF A PATIENT BY SHIFTING A LOCUS OF STIMULATION AND SYSTEM EMPLOYING THE SAME”, which is incorporated herein by reference. Optionally, the user interface 136 may permit the user to designate which electrodes 104-1 to 104-N are to stimulate (e.g., emit current pulses, in an anode state, in a cathode state), or not selected to stimulate (i.e., remain inactive or floating, i.e., “unused”), with respect to a potential stimulation target, to measure/sense tissue electrical parameters, or the like. As used herein “stimulation” refers to the application of an electrical signal to a target body tissue, regardless of the effect that signal is intended to produce. Additionally or alternatively, the external device 130 may access or download the electrical measurements from the memory 114 acquired by the sensing circuitry 126.

In some implementations, the external device 130 may permit operation of IPG 102 according to one or more spinal cord stimulation (SCS) programs or therapy applications to treat the patient. Each SCS program may include one or more sets of stimulation parameters of the pulse including pulse amplitude, stimulation level, pulse width, pulse frequency or inter-pulse period, pulse repetition parameter (e.g., number of times for a given pulse to be repeated for respective stimulation sets or stimsets during execution of program), biphasic pulses, monophasic pulses, etc. IPG 102 modifies its internal parameters in response to the control signals from the external device 130 to vary the stimulation characteristics of the stimulation therapy transmitted through the lead system 106A/106B to the tissue of the patient. Example neurostimulation (NS) systems, stimsets, and multi-stimset programs are set forth in U.S. Pat. No. 7,228,179, entitled “METHOD AND APPARATUS FOR PROVIDING COMPLEX TISSUE STIMULATION PATTERNS”, and International Patent Publication Number WO 2001/093953, entitled “NEUROMODULATION THERAPY SYSTEM”, which are incorporated hereinabove by reference.

Skilled artisans will recognize that in providing a stimulation signal to a target body tissue, an accumulation of continuous or net charge at the electrode/tissue interface (ETI) may occur, resulting in a residual voltage, which is highly undesirable as noted elsewhere in the present patent application. Because stimulation involves applying an electrical charge to body tissue. IMDs are required to ensure that a residual voltage at the ETI corresponding to each electrode are maintained under certain thresholds. Preferably, net charge at the ETI of an electrode needs to as close to zero as possible, i.e., the stimulation is charge balanced. Accordingly, in one arrangement, IPG 102 may include output coupling capacitors between the output circuits of the pulse generation/switching circuitry and the electrodes to block errant continuous direct current (DC) and serve as “passive” charge balancing components for the electrical signals being applied to the tissue. Accordingly, charge built up on the electrodes during stimulation may be offset by use of such output coupling capacitors (DC blocking stimulation capacitors), and may be discharged when delivery of a portion of the electrical signal is completed, e.g., typically after delivery of an individual pulse in a stimulation signal. A “discharge phase” may be observed for a period, for example, after a monophasic stimulation phase. In one arrangement, the stimulation phase and the discharge phase taken together may be considered a charge-balanced pulse in a signal comprising a plurality of such pulses. Even in such arrangements, however, there may be a gradual buildup of residual voltage across the DC blocking stimulation capacitors over time, depending on the frequency and type of pulsing schemes and associated stimsets used, in addition to the charge/voltage buildup at the ETI of an implantable lead system.

In accordance with the teachings of the present disclosure, embodiments described herein allow for DC blocking stimulation capacitors connected to the stimulation electrodes to continue to be used for maintaining patient safety while facilitating “Kelvin connection” paths using other unused/inactive stimulation electrodes and biosensing input AC-coupling capacitors on both sides of the ETI on an electrode-by-electrode basis in order to effectuate the measurement of residual voltages on individual electrodes. Turning to FIG. 1B, depicted therein is a pulse generator portion 100B having diagnostic circuitry and associated lead electrode capacitor arrangement according to an embodiment of the present disclosure. One skilled in the art will recognize upon reference hereto that various functionalities associated with example blocks shown as part of the pulse generator portion 100B may be distributed and/or integrated among one or more blocks, subsystems and/or modules described hereinabove with respect to FIG. 1A. Consistent with the description provided previously, a processing unit 152 having or associated with suitable digital control logic is operatively coupled to pulse control module 154, discharge module 156 and sensing/diagnostic circuitry 158 for facilitating various functionalities including but not limited to residual voltage measurements, active discharge cycling, electrode selection and configuration, etc. under appropriate programmatic control. An input/output interface block 160 is operatively coupled to a plurality of lead connectors 179-1 to 179-N interfaced with respective electrodes and associated ETI that may be represented as circuitry based on known or heretofore unknown charge-transfer mechanisms or models (not shown in this FIG.). Each lead connector 179-1 to 179-N may be provided with a DC blocking stimulation capacitor (C_{DC}) as well as an AC-coupling sense capacitor (C_{SENSE}) for facilitating direct current flow blocking and AC-coupling functionality with respect to the corresponding electrode that may be configured to operate as a stimulation node or a sensing node. By way of illustration, DC blocking stimulation capacitor C_{DC-1} 172-1 and sense capacitor C_{SENSE-1} 174-1 are coupled to lead connector 179-1 such that two interface terminals 177-1 and 176-1 are effectuated with respect to the lead circuitry of the interface block 160. Sense capacitor C_{SENSE-1} 174-1 is shunted across C_{DC-1} 172-1 such that an intermediate tap or node 175-1 is effectuated on the lead connector 179-1. Likewise, remaining lead connectors 179-N may be provided with respective C_{SENSE-N} 174-N shunted across C_{DC-N} 172-N to facilitate two interface terminals 177-N and 176-N for each corresponding lead electrode connector. As will be seen below, such an arrangement facilitates a Kelvin connection path via the sense capacitor interface terminal 176-N with respect to each active electrode for purposes of voltage measurement. Although the illustrated embodiment of FIG. 1B exemplifies an arrangement where each lead connector is provided with a corresponding sense capacitor, it should be appreciated that other arrangements may be realized within the scope of the present patent disclosure where not all lead connectors are coupled to respective sense capacitors.

Interface block 160 may include appropriate multiplexing and selection circuitry 162, anode/cathode/inactive electrode selection circuitry 164 and Kelvin connection (KC) mode selection circuitry 166 for effectuating various types of Kelvin connection schemes for measurement purposes while different electrodes of a lead system may be selectively configured for stimulation (e.g., anodic or cathodic stimulation), sensing, or designating unused/inactive states, etc., with appropriate electrical connections being made within an IPG device accordingly relative to the various components therein, under suitable programmatic control as needed. Example diagnostic circuitry 158 may comprise suitable analog-to-digital converter (ADC) circuitry configured for digital voltage measurement and associated signal processing using known voltage measurement techniques. As such, voltage measurement circuitry can be external and/or internal, on-board or off-board, and/or may be coupled to other measurement devices. Responsive to residual voltage measurements, active charge balancing may be effectuated by applying a discharge pulse of opposite polarity at a select electrode to reduce or eliminate the individual residual voltages of select electrodes by using discharge cycle module 156 in conjunction with switch circuitry under suitable programmatic control. Additional details regarding configuring lead electrodes as cathodes or anodes, either during stimulation or for discharging, may be found in may be found in U.S. Patent Application Publication No. 2009/0048643, entitled “METHOD FOR PROVIDING MULTIPLE VOLTAGE LEVELS DURING PULSE GENERATION AND IMPLANTABLE PULSE GENERATING EMPLOYING THE SAME”, which is hereby incorporated herein by reference.

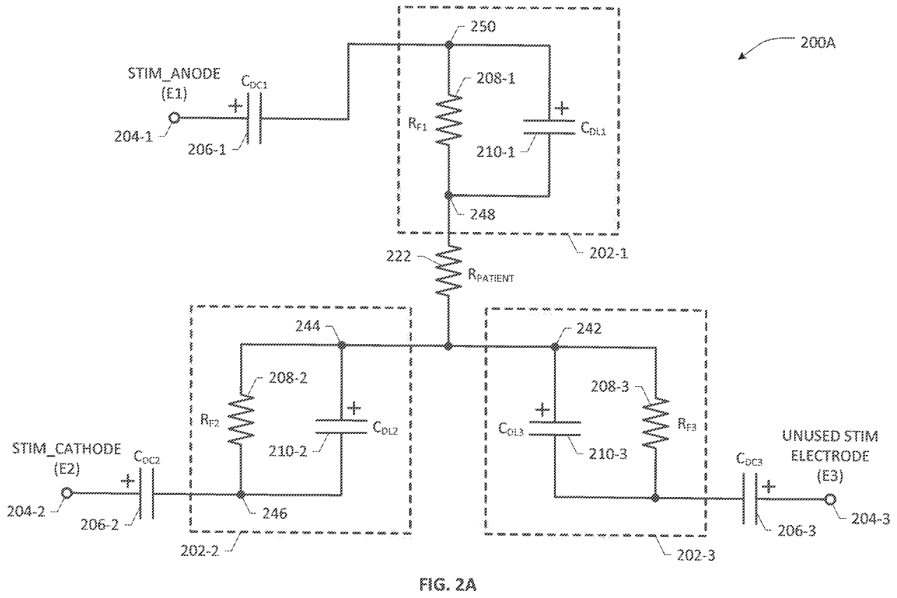

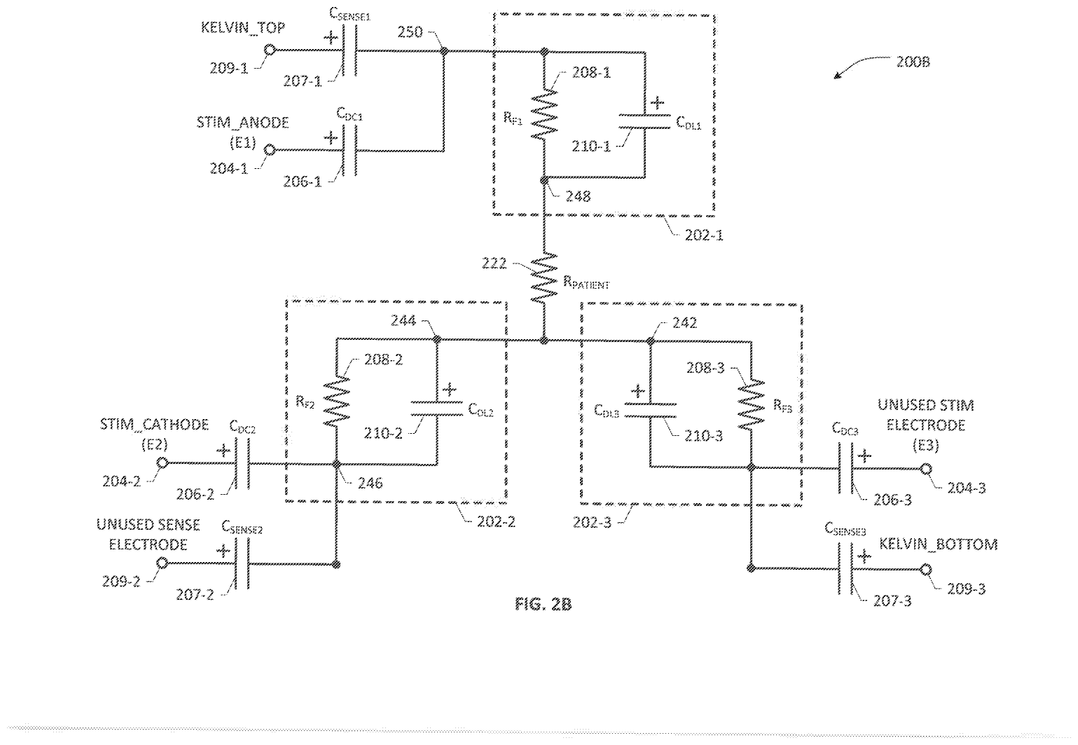

When an electrode is placed near tissue, current flow is determined by the flow of electrons in the electrode and flow of ions in the tissue. The electrode/electrolyte (i.e., tissue) interface (EEI or ETI; also sometimes referred to as electrode/patient interface or EPI) is typically modeled in accordance with a linear lumped element charge transfer model involving a series of lumped resistor elements coupled with a shunt capacitance that models the double layer of charge at the interface. In an illustrative ETI circuit representation as shown in FIG. 2A and FIG. 28, the solution resistance, R_{S}, is representative of the bulk electrolyte, which models the tissue or patient resistance, R_{PATIENT}. The double-layer capacitance or C_{DL} models the double layer of charge at the interface, which is coupled in parallel to a charge transfer resistance R_{F} across the interface. The charge transfer resistance, R_{F}, in parallel with the capacitance, C_{DL}, accounts for the conduction of charge through the interface, which can occur through various mechanisms, e.g., typically through oxidation-reduction reactions at the electrode for efficient operation of stimulation electrodes. Whereas more complex models of the electrode/tissue interface may be used, a charge transfer model is illustrated herein without necessarily being limited thereto for purposes of exemplifying how Kelvin connection paths may be advantageously effectuated for facilitating individual electrode RV measurements. It will be further appreciated that a “Kelvin connection” for purposes of the present patent disclosure is a circuit arrangement that allows avoiding voltage drops (thereby current flows) in circuit segments in a measurement or instrumentation circuit path that may interfere with or confound measurement variables. Furthermore, it will be apparent to one skilled in the art that embodiments disclosed herein facilitate such connection arrangements by unused DC blocking stimulation capacitor paths and/or AC-coupling sense capacitor paths in a number of schemes or combinations (collectively, “modes”) that may be selectively configured depending on a particular implementation.

FIG. 2A depicts an example circuit diagram 200A configured for facilitating RV measurements using one type of Kelvin connection path according to an embodiment of the present disclosure wherein three electrodes (E1, E2 and E3) and respective tissue interfaces 202-1 to 202-3 are shown by way of example. Illustratively, electrodes E1 and E2 are configured as stimulation anode and cathode, respectively, with electrode E3 being left unused or inactive. Each electrode is provided with a respective DC blocking stimulation capacitor C_{DC}, which facilitates a terminal or node with respect to an interface block coupled to suitable diagnostic/sense circuitry as described previously. Further, each ETI 202-1 to 202-3 is exemplified by a corresponding C_{DL} 210-1 to 210-3 coupled in parallel to respective charge transfer resistance R_{F} 208-1 to 208-3, that is in series connection with the bulk patient resistance R_{PATIENT} 222 effectively disposed between a pair of the electrodes in any applicable combination. Because E3 is configured as an unused electrode for stimulation, its DC blocking stimulation capacitor C_{DC3} 206-3 is kept in a discharged state, which allows the associated terminal 204-3 to be used in a Kelvin connection path with respect to any other electrode terminals in a measurement circuit loop. For example, a measurement loop between terminal 204-2 of cathode-active electrode E2 and terminal 204-3 of unused electrode E3 can be used to measure a residual voltage comprising a sum of any voltage buildup across C_{DC2} 206-2 and a voltage buildup across C_{DL2} 210-2 because terminal 204-3 is at the same level as internal nodes 242 and 244 of the circuit arrangement 200A. In a typical DBS implementation where C_{DC} capacitances are substantially larger than the C_{DL} capacitances (e.g., by several orders of magnitude), the charge buildup on the DC blocking stimulation capacitors may be small enough that it may be ignored in estimating the RV measurement across C_{DL} associated with the selected active electrode, e.g., E2. In such a scenario, the total RV measurement may therefore be treated as being sufficiently close to the RV component due to the buildup associated with C_{DL2} 210-2. On the other hand, in typical SCS implementations, the differences between C_{DL} and C_{DC} capacitances are usually less than one order of magnitude (e.g., around seven times). Accordingly, in such an application, the charge buildup on SCS DC blocking stimulation capacitors (C_{DC}) cannot be ignored for RV measurements as readily.

In similar fashion, an RV measurement loop between terminal 204-1 of electrode E1 (configured as an anode stimulation node) and terminal 204-3 of unused electrode E3 can be effectuated in order obtain a cumulative RV measurement after stimulation comprising an RV component representing voltage buildup across C_{DC1} 206-1 and voltage buildup across C_{DL1} 210-1 since terminal 204-3 is at the same voltage level as internal nodes 242 and 248 (because outside of stimulation there is little current flow in the inactive electrode path through the bulk tissue resistance R_{PATIENT} 222; however, the inactive electrode is most generally used as a Kelvin connection only when there is no stimulation nor discharge current flowing through the patient/tissue, although there can be exceptions). Further, the cumulative RV measurement may be treated as a reasonable approximation of the RV buildup after stimulation across C_{DL1} 210-1 since C_{DC1} 206-1 is typically much larger than C_{DL1} 210-1 in certain applications, as noted previously. An example implementation of the circuit arrangement 200A may comprise C_{DC} capacitances around 20-30 μF whereas the C_{DL} capacitances may be around 0.1-3.0 μF. Skilled artisans will also recognize that the C_{DC} capacitance values may be even lower, e.g., around 10-15 μF, especially in smaller physical form factor implementations.

Accordingly, a Kelvin connection path effectuated via the C_{DC} terminal of an inactive electrode of an implantable lead system may be used for obtaining an RV measurement associated with any of the active electrodes of the lead system, albeit with an added RV component associated with the C_{DC} capacitor corresponding to the selected active electrode. To separate this additional RV component from the RV measurement path, an AC-coupling sense capacitor path of an active electrode may be used in example embodiments as a Kelvin connection path at the other end of the measurement loop in conjunction with a Kelvin connection path at an inactive electrode as set forth above. In further embodiments, an inactive electrode may also be provided with an AC-coupling sense capacitor path (which is a likely implementation scenario since it is preferable to manufacture identical electrodes in a lead system that can be selectively and dynamically configured depending on a particular stimulation application and associated stimset variations). In such embodiments, an alternative Kelvin connection path may be established at the inactive electrode in addition to the inactive DC blocking stimulation C_{DC} capacitor path thereat. One skilled in the art will therefore readily appreciate that a number of Kelvin connection modes may be effectuated in an example IMD/IPG system depending on the various AC-coupling and/or DC blocking stimulation capacitor arrangements provided with respect to the electrodes of a lead system and/or how the different electrodes and corresponding capacitor arrangements are selectively configured. For example, where a subset of the electrodes are configured to be active, the remaining electrodes (one or more of the rest of the electrodes) may be disposed as inactive electrodes, out of which any one particular electrode may be configured as one end of a Kelvin connection path with respect to an RV measurement loop. Such a Kelvin connection path may be effectuated via the selected inactive node's DC blocking stimulation capacitor path or via its AC-coupling sense capacitor path, as noted above. In an additional/alternative embodiment, one of the electrodes of a lead system may be designated or dedicated to operate as a Kelvin connection terminal for effectuating RV measurements with respect to any one of the active electrodes of the lead system according to the teachings of the present patent disclosure.

Turning to FIG. 2B, depicted therein is an example circuit diagram 200B configured to exemplify one or more of the foregoing embodiments for facilitating RV measurements using different types and/or combinations of Kelvin connection paths according to the present patent disclosure. Similar to the arrangement 200A illustrated in FIG. 2A, circuit arrangement 200B of FIG. 2B exemplifies three electrodes, E1-E3, each shown with corresponding ETI circuit representations 202-1 to 202-3 coupled to bulk patient resistance R_{PATIENT} 222 in a “star” configuration. Further, electrodes E1 and E2 are illustrated as active stimulation nodes while electrode E3 is left as an inactive/unused electrode as before. By way of illustration, each electrode is provided with corresponding biosensing input terminal 209-1 to 209-3, effectuated via respective AC-coupling capacitors C_{SENSE1} 207-1 to C_{SENSE3} 207-3 that are coupled in parallel to the respective C_{DC1} 206-1 to C_{DC3} 206-3 capacitors. In one embodiment, the AC-coupling capacitors C_{SENSE1} 207-1 to C_{SENSE3} 207-3 may be implemented as low capacitance components (e.g., around 0.1 μF), which may be maintained to be readily kept in a discharged state (e.g., because no stimulation current will ever flow through such capacitors). Accordingly, voltage levels at the AC-coupling capacitors C_{SENSE1} 207-1 to C_{SENSE3} 207-3 of electrodes E1-E3 are always near or close to 0 V (or some other reference potential), which can facilitate respective Kelvin connection paths for measuring the electrode residual voltages in connection with a select RV measurement loop depending on which electrode's RV is being measured. For example, C_{SENSE1} 207-1 terminal 209-1 may be deemed a “Kelvin_Top” terminal which is at the same potential as internal node 250 with respect to ETI 202-1 of electrode E1. By utilizing C_{SENSE3} 207-3 terminal 209-3 of the unused electrode E3 as a “Kelvin_Bottom” terminal (which is at the same potential as internal node 242), an RV measurement after stimulation across C_{DL1} 210-1 may be obtained in a manner similar to the RV measurement process discussed above. Further, the C_{DC3} terminal 204-3 of the unused electrode E3 may also be used in conjunction with the C_{SENSE1} 207-1 terminal 209-1 operating as the “Kelvin_Top” terminal in an alternative embodiment, as previously described. Skilled artisans will recognize this alternative Kelvin connection path may be beneficial to use if the biosensing AC-coupling C_{SENSE3} 207-3 terminal 209-3 associated with electrode E3 is already in use for biosensing and it is required that the sensing activity from electrode E3 remain undisturbed. Likewise, RV buildup at other active electrodes (i.e., across respective C_{DL} capacitances) may be measured after stimulation by using corresponding C_{SENSE} terminal inputs in conjunction with either of the Kelvin connection paths available at the unused electrode E3 in a similar manner.

Skilled artisans will appreciate that whereas example Kelvin connection paths illustrated above involve a pair of electrodes across the EPI interface with suitable capacitor terminals operating as Kelvin connection terminals, additional and/or alternative embodiments according to the teachings of the present invention may also involve any combination of any subset of the active electrodes and any subset of the unused/linactive electrodes in a Kelvin connection path on the either side of the EPI interface for an RV measurement, with appropriate capacitor terminal connections as described herein, mutatis mutandis.

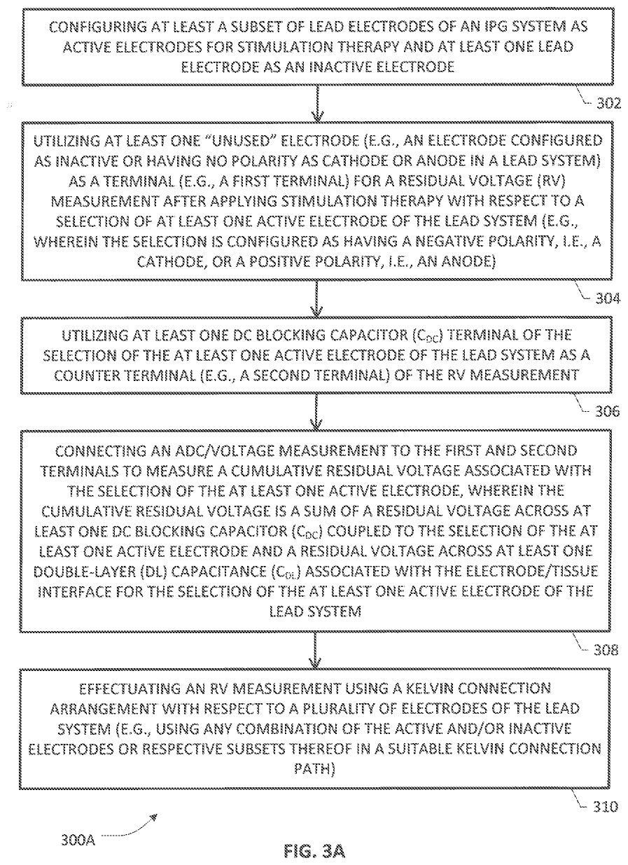

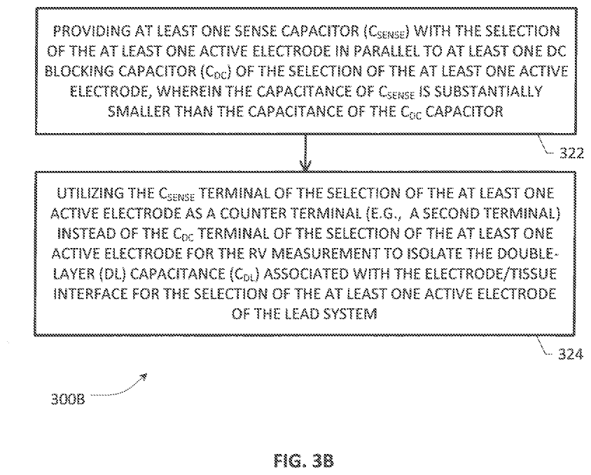

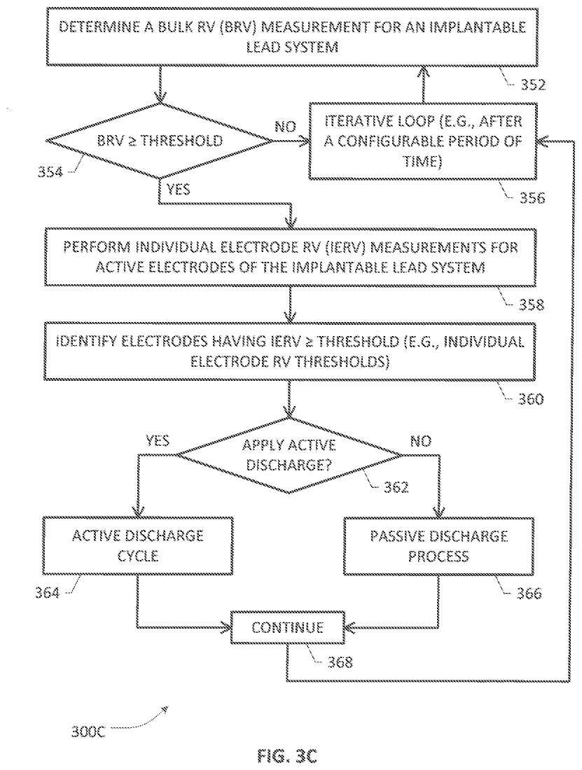

FIGS. 3A-3C depict flowcharts illustrative of blocks, steps and/or acts that may be (re)combined in one or more arrangements for effectuating RV measurements according to an embodiment of the present disclosure. Example Process 300A commences with respect to configuring at least a subset of lead electrodes of an IPG system as active electrodes and at least one lead electrode as an inactive electrode (block 302). At block 304, at least one “unused” electrode (e.g., an electrode configured as inactive or having no polarity as cathode or anode in a lead system) may be designated, dedicated or otherwise utilized as a terminal (e.g., a first terminal) for a residual voltage (RV) measurement with respect to a selection of at least one active electrode of the lead system (e.g., wherein the selection of at least one electrode is configured as having a negative polarity, i.e., a cathode, or a positive polarity, i.e., an anode). In one embodiment, at least one DC blocking stimulation capacitor (C_{DC}) terminal of the selection of the at least one active electrode of the lead system is utilized as a counter terminal (e.g., a second terminal) of the RV measurement (block 306) in a Kelvin connection arrangement. A diagnostic module or circuitry including an ADC/voltage measurement system is electrically connected to the first and second terminals to effectuate a measurement loop for measuring a cumulative residual voltage associated with the selection of the at least one active electrode (block 308). As noted previously, such a measurement results in the cumulative residual voltage as being a sum of a residual voltage across the at least one DC blocking stimulation capacitor (C_{DC}) coupled to the selection of the at least one active electrode and a residual voltage across at least one double-layer (DL) capacitance (C_{DL}) associated with the electrode/tissue interface for the selection of the at least one electrode of the lead system, the RV component across the one or more C_{DC} capacitors may be neglected in some example embodiments. Skilled artisans will therefore appreciate that an RV measurement using a Kelvin connection arrangement according to an embodiment of the present invention may be effectuated with respect to a plurality of electrodes of the lead system, e.g., any combination of how active/inactive electrodes could be configured and used as appropriate Kelvin connection terminals in an RV measurement (block 310).

Processes 300B and/or 300C exemplify additional and/or alternative embodiments involving or modifying at least a portion of the foregoing process. At block 322 of process 300B, at least one sense capacitor (C_{SENSE}) is provided with a selection of at least one active electrode in parallel to at least one DC blocking stimulation capacitor (C_{DC}) of the selection of the at least one active electrode, wherein the capacitance of C_{SENSE} is substantially smaller than the capacitance of than the C_{DC} capacitor. At block 324, the C_{SENSE} terminal of the selection of the at least one active electrode is used as a counter terminal (e.g., a second terminal) instead of the at least one C_{DC} terminal of the selection of the at least one active electrode for the RV measurement to isolate at least one double-layer (DL) capacitance (C_{DL}) associated with the electrode/tissue interface for the selection of the at least one active electrode of the lead system. Accordingly, a more accurate RV measurement of the electrode RV buildup, i.e., across the C_{DL} capacitance, may be obtained in this embodiment.

Process 300C of FIG. 3C is illustrative of an application scenario where one or more RV measurement processes of the present patent disclosure may be advantageously implemented. At block 352, a bulk residual voltage (BRV) measurement may be obtained by known or heretofore unknown techniques that measure or obtain an overall voltage buildup associated with an entire array of electrodes of an implantable lead system. At block 354, a determination may be made whether the measured/stored BRV value is greater than or equal to a threshold (e.g., a first threshold or BRV threshold). If so, RV measurements associated with individual electrodes of the lead system may be performed using any Kelvin connection mode(s) as described herein (block 358). Otherwise, an iterative loop may be executed, e.g., based on a dynamically configurable of time elapsed, modulated responsive to triggering events or notifications due to or caused by external events, etc., until a next BRV measurement is taken, as set forth at block 356. At block 360, a determination may be made to determine or otherwise identify if any individual electrode has an RV greater than or equal to a threshold (e.g., a second threshold or individual electrode RV (IERV) threshold). Upon identifying electrodes having an RV≥IERV threshold, a determination as to applying an active discharge cycle may be made with respect to the electrodes, e.g., on electrode-by-electrode basis (block 362). If active discharge pulsing is selected, appropriate discharge pulses (e.g., positive or negative polarity pulses) may be provided to the respective electrodes (block 364). Otherwise, a passive discharging scheme may be allowed to take place, e.g., pursuant to applicable stimulation set protocols or other criteria (block 366). Thereafter, the process flow may continue to the iterative loop process 356, e.g., until further diagnostic measurements are needed, as set forth above.

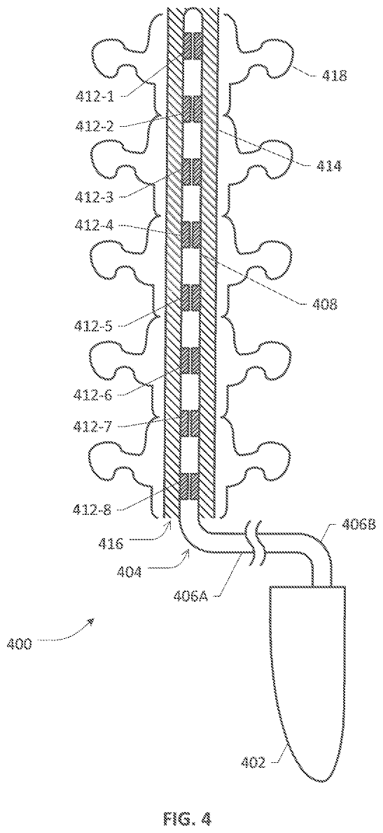

FIG. 4 illustrates an example spinal cord stimulation (SCS) therapy application 400 involving a pulse generator or IMD 402 and associated lead system 404 having a plurality of electrodes 412-1 to 412-8 wherein diagnostic RV measurements may be obtained using an embodiment of the present disclosure. Preferably, the lead system 404 comprises a lead body 406A/B coupled to an implantable lead 408 that may be positioned at a desired target position in an epidural space 416 defined by a plurality of vertebrae of a patient so as to be in close proximity to a nerve tissue of interest, e.g., a spinal cord 414. The implantable lead 408 includes eight electrodes 412-1 to 412-8, which may comprise ring electrodes, segmented or split electrodes, and the like that may be separated from one another by equal or unequal portions of encapsulating material. The implantable lead 408 is connected via lead body 406A/406B to the pulse generator or IPG 402 that includes at least an embodiment of a Kelvin connection scheme of the present disclosure that is configured to be operative with suitable diagnostic circuitry. As noted previously, at least a subset of the electrodes 412-1 to 412-8 may be selectively energized, i.e., stimulated, whereupon suitable RV measurements may be taken using the Kelvin connection scheme. For example, in one embodiment electrodes 412-1, 412-4 and 412-8 may be programmed as cathodes or anodes for operation in conjunction with the case or can of the IPG 402 for providing current stimulation to effectuate an electric field that is spatially distributed over entire target portion of the spinal cord 414. An unused electrode, e.g., electrode 412-5, may be used to establish a Kelvin connection path on the inactive side of the measurement loop with respect to any of the selected active electrodes 412-1, 412-4 and 412-8 for measuring RVs associated therewith.

In one embodiment, the diagnostic circuitry of IPG/IMD 402 may therefore be configured to perform, under programmatic control, the following: utilize one of a direct current (DC) blocking stimulation capacitor (C_{DC}) terminal and an alternating current (AC) coupling sense capacitor (C_{SENSE}) terminal of an inactive electrode, e.g., electrode 412-5 of the implantable lead system 404 as a first Kelvin connection terminal for a residual voltage measurement with respect to a select active electrode, e.g., electrode 412-4, of the implantable lead system 404; utilize a terminal of an alternating current (AC) coupling sense capacitor (C_{SENSE}) coupled to the select active electrode 412-4 as a second Kelvin connection terminal for the residual voltage (RV) measurement; and electrically couple a voltage measurement circuit to the first and second Kelvin connection terminals to measure a residual voltage associated with the select active electrode that is accumulated across a double-layer (DL) capacitance (C_{DL}) associated with an electrode/tissue interface of the select active electrode.

In a further embodiment, the diagnostic circuitry may be configured to effectuate the RV measurement responsive to at least one of following: (i) determining that a bulk RV associated with the implantable lead system 404 is greater than or equal to a bulk threshold value; (ii) a period of time after applying stimulation therapy to the biological tissue, e.g., tissue 414 (for instance, to determine how much discharge is needed; or an RV measurement could be done after stimulation and discharge, to determine how effective was the discharge cycle at removing the residual voltage); and/or (iii) determining that an electromagnetic field perturbation or interference has been encountered by the biological tissue (e.g., due to magnetic resonance imaging or MRI, or any other type of scanning that causes an electromagnetic field near or in the vicinity of the IMD); or in any combination thereof. In a still further embodiment, a discharge cycle module of IPG/IMD 402 (shown particularly in FIG. 1B as module 156) may be operative for applying an active discharge pulse to the select electrode 412-4 responsive to determining at least one of following: (i) the bulk RV associated with the implantable lead system 404 is greater than or equal to the bulk RV threshold value; and/or (ii) the individual electrode RV measurement associated with the select active electrode 412-4 of the implantable lead system is greater than or equal to the individual electrode threshold value.

Based on the foregoing Detailed Description, skilled artisans will recognize that embodiments of the present patent disclosure may be advantageously configured to allow unused stimulation electrode DC-blocking stimulation capacitors and/or biosensing electrode input AC-coupling capacitors for establishing one or more Kelvin connection pathways so as to enable the monitoring of the voltage and state of charge of the electrode/patient interfaces for any of the individual electrodes in an implantable medical device. With the ability to measure and monitor the residual voltage and state of charge on each of the individual electrode interfaces to the patient (e.g., by connecting the non-patient sides of the “Kelvin connection” components to an onboard A/D converter in the IMD), it can be determined how to optimally discharge the electrodes after stimulation (e.g., based on an estimation of decay time constant) in order to maintain their respective steady-state interface voltages and charge states at very low and safe levels. It will be appreciated that example embodiments are particularly advantageous for providing accurate residual voltage measurement results when there are no electrode stimulation or discharge currents flowing through the patient (in order to ensure that there is no voltage drop across the R_{PATIENT} portion of the stimulation load network), as exemplified in the circuit arrangements of FIGS. 2A and 2B.

The capability of being able to monitor the interface to the patient at each individual stimulation electrode can provide two key benefits in an implantable biostimulation system: (i) help maintain the electrode interfaces in low steady-state voltage conditions, which are highly beneficial to the long-term integrity of the electrodes because the incidence/rate of detrimental electrochemical reactions (e.g., reduction/oxidation reactions) is reduced; and (ii) help ensure the comfort and safety of the patient by reducing peak RV buildup.

In addition to the foregoing reliability and safety benefits, example embodiments of the present disclosure may also help to reduce IPG/IMD battery current, which would enhance the battery longevity of implantable biological devices. Still further, example embodiments having the “Kelvin connection” capability can also provide an expanded diagnostic feature set to the implantable biostimulation system, which could enable researchers, clinicians, and technicians, etc. to more extensively investigate the therapeutic benefits of novel stimulation waveforms for patients, especially in correlation to the amounts of residual voltage left on the electrode/patient interfaces after stimulation.

In the above-description of various embodiments of the present disclosure, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of this specification and the relevant art and may not be interpreted in an idealized or overly formal sense expressly so defined herein.

At least some example embodiments are described herein with reference to one or more circuit diagrams/schematics, block diagrams and/or flowchart illustrations. It is understood that such diagrams and/or flowchart illustrations, and combinations of blocks in the block diagrams and/or flowchart illustrations, can be implemented by any appropriate circuitry configured to achieve the desired functionalities. Accordingly, example embodiments of the present disclosure may be embodied in hardware and/or in software (including firmware, resident software, micro-code, etc.) operating in conjunction with suitable processing units or microcontrollers, which may collectively be referred to as “circuitry,” “a module” or variants thereof. An example processing unit or a module may include, by way of illustration, a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Array (FPGA) circuits, any other type of integrated circuit (IC), and/or a state machine, as well as programmable system devices (PSDs) employing system-on-chip (SoC) architectures that combine memory functions with programmable logic on a chip that is designed to work with a standard microcontroller. Example memory modules or storage circuitry may include volatile and/or nonvolatile memories such as, e.g., random access memory (RAM), electrically erasable/programmable read-only memories (EEPROMs) or UV-EPROMS, one-time programmable (OTP) memories, Flash memories, static RAM (SRAM), etc.

Further, in at least some additional or alternative implementations, the functions/acts described in the blocks may occur out of the order shown in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Furthermore, although some of the diagrams include arrows on communication paths to show a primary direction of communication, it is to be understood that communication may occur in the opposite direction relative to the depicted arrows. Finally, other blocks may be added/inserted between the blocks that are illustrated.

It should therefore be clearly understood that the order or sequence of the acts, steps, functions, components or blocks illustrated in any of the flowcharts depicted in the drawing Figures of the present disclosure may be modified, altered, replaced, customized or otherwise rearranged within a particular flowchart, including deletion or omission of a particular act, step, function, component or block. Moreover, the acts, steps, functions, components or blocks illustrated in a particular flowchart may be inter-mixed or otherwise inter-arranged or rearranged with the acts, steps, functions, components or blocks illustrated in another flowchart in order to effectuate additional variations, modifications and configurations with respect to one or more processes for purposes of practicing the teachings of the present patent disclosure.

Although various embodiments have been shown and described in detail, the claims are not limited to any particular embodiment or example. None of the above Detailed Description should be read as implying that any particular component, element, step, act, or function is essential such that it must be included in the scope of the claims. Reference to an element in the singular is not intended to mean “one and only one” unless explicitly so stated, but rather “one or more.” Moreover, the terms “first,” “second,” and “third,” etc. employed in reference to elements or features are used merely as labels, and are not intended to impose numerical requirements, sequential ordering or relative degree of significance or importance on their objects. All structural and functional equivalents to the elements of the above-described embodiments that are known to those of ordinary skill in the art are expressly incorporated herein by reference and are intended to be encompassed by the present claims. Accordingly, those skilled in the art will recognize that the exemplary embodiments described herein can be practiced with various modifications and alterations within the spirit and scope of the claims appended below.

1. An implantable medical device, comprising:
a power supply module;
a processing unit;
an implantable lead system comprising a plurality of electrodes adapted to stimulate a biological tissue responsive to instructions generated by the processing unit in association with a pulse switching module, the plurality of electrodes including at least one inactive electrode and at least one active electrode; and
diagnostic circuitry coupled to the processing unit and the pulse switching module, the diagnostic circuitry configured to:
utilize one of at least one of a direct current (DC) blocking stimulation capacitor (C_{DC}) terminal and at least one of an alternating current (AC) coupling sense capacitor (C_{SENSE}) terminal of the at least one inactive electrode of the implantable lead system as a first Kelvin connection terminal for a residual voltage measurement with respect to a selection of the at least one active electrode of the implantable lead system;
utilize a terminal of at least one of an alternating current (AC) coupling sense capacitor (C_{SENSE}) coupled to the selection of the at least one active electrode as a second Kelvin connection terminal for the residual voltage (RV) measurement; and
electrically couple a voltage measurement circuit to the first and second Kelvin connection terminals to measure a residual voltage associated with the selection of the at least one active electrode that is accumulated across at least one double-layer (DL) capacitance (C_{DL}) associated with an electrode/tissue interface of the selection of the at least one active electrode.

2. The implantable medical device as recited in claim 1, wherein the diagnostic circuitry is further configured to operate with a mode selector for utilizing at least one direct current (DC) blocking stimulation capacitor (C_{DC}) terminal of the selection of the at least one active electrode of the implantable lead system as a second terminal for the RV measurement instead of the second Kelvin connection terminal such that the residual voltage of the selection of the at least one active electrode is obtained as a sum of an RV component accumulated across at least one C_{DC} capacitor coupled to the selection of the at least one active electrode and an RV component accumulated across at least one C_{DL} capacitance associated with the electrode/tissue interface of the selection of the at least one active electrode.

3. The implantable medical device as recited in claim 2, wherein the diagnostic circuitry is configured to effectuate the RV measurement responsive to at least one of following: (i) determining that a bulk RV associated with the implantable lead system is greater than or equal to a bulk threshold value; (ii) a period of time after applying stimulation therapy to the biological tissue; and (iii) determining that an electromagnetic interference has been encountered by the biological tissue.

4. The implantable medical device as recited in claim 3, further comprising:
a discharge cycle module operative with the pulse switching module for applying an active discharge pulse to the selection of the at least one active electrode responsive to determining at least one of following: (i) the bulk RV associated with the implantable lead system is greater than or equal to the bulk threshold value; and (ii) the RV measurement associated with the selection of the at least one active electrode of the implantable lead system is greater than or equal to an individual electrode threshold value.

5. The implantable medical device as recited in claim 4, wherein the C_{SENSE} capacitors respectively coupled to the selection of the at least one active electrode and the at least one inactive electrode each have substantially smaller capacitances than capacitances of the respective C_{DC} capacitors coupled to the selection of the at least one active electrode and the at least one inactive electrode.

6. The implantable medical device as recited in claim 5, wherein the at least one inactive electrode comprises a dedicated electrode of the implantable lead system for facilitating at least one Kelvin connection path for measuring individual electrode RV values respectively associated with one or more active electrodes of the implantable lead system.

7. The implantable medical device as recited in claim 6, wherein the selection of the at least one active electrode is configured as one of a cathode to provide cathodic stimulation to the biological tissue and an anode to provide anodic stimulation to the biological tissue.

8. A pulse generator configured to supply stimulation to a biological tissue, the pulse generator comprising:
a power supply module;
a processing unit;
an implantable lead system comprising a plurality of electrodes adapted to stimulate the biological tissue responsive to instructions generated by the processing unit in association with a pulse switching module, the plurality of electrodes including at least one inactive electrode and at least one active electrode; and
diagnostic circuitry coupled to the processing unit and the pulse switching module, the diagnostic circuitry configured to:
utilize at least one direct current (DC) blocking stimulation capacitor (C_{DC}) terminal of the at least one inactive electrode of the implantable lead system as a first terminal for a residual voltage (RV) measurement with respect to a select active electrode of the implantable lead system;
utilize at least one direct current (DC) blocking stimulation capacitor (C_{DC}) terminal of the selection of the at least one active electrode of the implantable lead system as a second terminal for the RV measurement; and
electrically couple a voltage measurement circuit to the first and second terminals to measure a residual voltage associated with the selection of the at least one active electrode, wherein the first terminal is operative to provide a Kelvin connection path with respect to the at least one inactive electrode for the voltage measurement circuit such that the residual voltage is obtained as a sum of a first residual voltage (RV) component accumulated across at least one C_{DC} capacitor coupled to the selection of the at least one active electrode and a second residual voltage (RV) component accumulated across at least one double-layer (DL) capacitance (C_{DL}) associated with an electrode/tissue interface of the selection of the at least one active electrode.

9. The pulse generator as recited in claim 8, wherein the diagnostic circuitry is further configured to operate with a mode selector for utilizing a terminal of at least one alternating current (AC) coupling sense capacitor (C_{SENSE}) coupled to the at least one inactive electrode as the first terminal instead of the C_{DC} terminal of the at least one inactive electrode, the C_{SENSE} terminal coupled to the at least one inactive electrode operating as an alternative Kelvin connection path with respect to the at least one inactive electrode for measuring the residual voltage of the selection of the at least one active electrode comprising the sum of the first and second RV components.

10. The pulse generator as recited in claim 9, wherein the diagnostic circuitry is further configured to operate with the mode selector for utilizing a terminal of at least one alternating current (AC) coupling sense capacitor (C_{SENSE}) coupled to the selection of the at least one active electrode as the second terminal instead of the C_{DC} terminal of the selection of the at least one active electrode, the C_{SENSE} terminal coupled to the selection of the at least one active electrode operating as an active side Kelvin connection path with respect to the selection of the at least one active electrode, to obtain a voltage measurement comprising only the RV component accumulated across at least one C_{DL} capacitance associated with the electrode/tissue interface of the selection of the at least one active electrode regardless of which Kelvin connection path at the at least one inactive electrode is used.

11. The pulse generator as recited in claim 9, wherein the diagnostic circuitry is configured to effectuate the RV measurement responsive to at least one of following: (i) determining that a bulk RV associated with the implantable lead system is greater than or equal to a bulk threshold value; (ii) a period of time after applying stimulation therapy to the biological tissue; and (iii) determining that an electromagnetic interference has been encountered by the biological tissue.

12. The pulse generator as recited in claim 11, further comprising:
a discharge cycle module operative with the pulse switching module for applying an active discharge pulse to the selection of the at least one active electrode responsive to determining at least one of following: (i) the bulk RV associated with the implantable lead system is greater than or equal to the bulk threshold value; and (ii) the RV measurement associated with the selection of the at least one active electrode of the implantable lead system is greater than or equal to an individual electrode threshold value.

13. A method operative with a pulse generator configured to supply stimulation to a biological tissue, the method comprising:
configuring at least one of a plurality of electrodes of an implantable lead system of the pulse generator as an active electrode operative to apply stimulation therapy to the biological tissue responsive to instructions generated by a processing unit in association with a pulse switching module;
configuring at least one of the plurality of electrodes of the implantable lead system as an inactive electrode;
utilizing at least one direct current (DC) blocking stimulation capacitor (C_{DC}) terminal of the at least one inactive electrode of the implantable lead system as a first terminal for a residual voltage measurement with respect to a selection of the at least one active electrode of the implantable lead system;
utilizing at least one direct current (DC) blocking stimulation capacitor (C_{DC}) terminal of the selection of the at least one active electrode of the implantable lead system as a second terminal for the RV measurement; and
electrically coupling a voltage measurement circuit to the first and second terminals to measure a residual voltage associated with the selection of the at least one active electrode, wherein the first terminal is operative to provide a Kelvin connection path with respect to the at least one inactive electrode for the voltage measurement circuit such that the residual voltage is obtained as a sum of a first residual voltage (RV) component accumulated across at least one C_{DC} capacitor coupled to the selection of the at least one active electrode and a second residual voltage (RV) component accumulated across at least one double-layer (DL) capacitance (C_{DL}) associated with an electrode/tissue interface of the selection of the at least one active electrode.

14. The method as recited in claim 13, further comprising:
selecting a Kelvin connection mode in a first setting;
utilizing a terminal of at least one alternating current (AC) coupling sense capacitor (C_{SENSE}) coupled to the at least one inactive electrode as the first terminal instead of the C_{DC} terminal of the at least one inactive electrode, the C_{SENSE} terminal coupled to the at least one inactive electrode operating as an alternative Kelvin connection path with respect to the at least one inactive electrode; and
obtaining the residual voltage of the selection of the at least one active electrode as the sum of the first and second RV components.

15. The method as recited in claim 14, further comprising:
selecting the Kelvin connection mode in a second setting;
utilizing a terminal of at least one alternating current (AC) coupling sense capacitor (C_{SENSE}) coupled to the selection of the at least one active electrode as the second terminal instead of the C_{DC} terminal of the selection of the at least one active electrode, the C_{SENSE} terminal coupled to the selection of the at least one active electrode operating as an active side Kelvin connection path with respect to the selection of the at least one active electrode; and
obtaining a voltage measurement comprising only the RV component accumulated across at least one C_{DL} capacitance associated with the electrode/tissue interface of the selection of the at least one active electrode regardless of which Kelvin connection path at the at least one inactive electrode is used.

16. The method as recited in claim 15, wherein the selection of the at least one active electrode is configured as one of a cathode to provide cathodic stimulation to the biological tissue and an anode to provide anodic stimulation to the biological tissue.

17. The method as recited in claim 16, further comprising:
effectuating the RV measurement responsive to at least one of following: (i) determining that a bulk RV associated with the implantable lead system is greater than or equal to a bulk threshold value; (ii) a period of time after applying stimulation therapy to the biological tissue; and (iii) determining that an electromagnetic interference has been encountered by the biological tissue.

18. The method as recited in claim 17, further comprising:
applying an active discharge pulse to the selection of the at least one electrode upon determining at least one of following: (i) the bulk RV associated with the implantable lead system is greater than or equal to the bulk threshold value; and (ii) the RV measurement associated with the selection of the at least one active electrode of the implantable lead system is greater than or equal to an individual electrode threshold value.

19. The method as recited in claim 18, further comprising:
providing the C_{SENSE} capacitors respectively coupled to the selection of the at least one active electrode and the at least one inactive electrode to each have substantially smaller capacitances than capacitances of the respective C_{DC} capacitors coupled to the selection of the at least one active electrode and the at least one inactive electrode.

20. The method as recited in claim 19, further comprising:
providing the at least one inactive electrode as a dedicated electrode of the implantable lead system for facilitating at least one Kelvin connection path for measuring individual electrode RV values respectively associated with one or more active electrodes of the implantable lead system.
